(19) United States
(12) Patent Application Publication
(10) Pub. No.: US 2004/0220495 A1
(43) Pub. Date: Nov. 4, 2004

(54) Diagnostic device

(75) Inventors: Nicholas Cahir, Cambridge (GB); Michelle L. Pataki, Elsternwick (AU)

Correspondence Address: IRELL & MANELLA LLP, 1800 AVENUE OF THE STARS, SUITE 900, LOS ANGELES, CA, 90067, US

(73) Assignee: Norwood Abbey Ltd., Chelsea Heights (AU)

(21) Appl. No.: 10/402,343

(22) Filed: Mar. 28, 2003

Related U.S. Application Data
(63) Continuation of application No. PCT/AU01/01223, filed on Sep. 28, 2001.

(51) Int. Cl. A61B 10/00

(52) U.S. Cl. 600/562

(57) ABSTRACT

The invention relates to a diagnostic device 10 for collecting and analysing a biological sample comprising, an energy source providing means for perforating, ablating and/or altering the stratum corneum of an area of skin from which the biological sample is to be collected; a housing 21 for receiving at least one test strip 22, the test strip being adapted to collect biological sample from the perforated, ablated and/or altered area of skin; and analysing means for conducting diagnostic analysis of the collected sample. The invention also relates to a cartridge 20 containing a plurality of test strips 22 for collecting a biological sample, each test strip comprising an absorbent portion 35 for absorbing the biological sample at an area of skin which has had applied thereto an energy source to perforate, ablate or alter the stratum corneum of the skin, wherein said test strips are adapted to facilitate transmission of the energy source to the skin.

This application is claims benefits of PCT/AU01/01223 filed Sep. 28, 2001, which is a PCT filing of AU application PR 0440 filed Sep. 28, 2000, each of which is incorporated herein by reference.

The present invention relates to a diagnostic device for use in the medical field. More particularly, the invention relates to a device which, in use, perforates, ablates or alters the stratum corneum layer of the skin and subsequently or simultaneously performs a diagnostic test on fluids, gases and/or biomolecules removed from or permeating through the skin following the perforation, ablation or alteration.

Traditional methods for the collection of small quantities of fluids or gases from a patient utilize mechanical puncture of the skin with a sharp device such as a metal lancet or needle. These procedures have many drawbacks, two of which are the possible infection of health-care workers or the public at large with the device used to perforate the skin, and the costly handling and disposal of biologically hazardous waste.

When skin is punctured with a sharp device such as a metal lancet or needle, biological waste is created in the form of the “sharp” which is contaminated by the patients blood and/or tissue. If the patient is infected with any number of blood-born agents, such as human immunodeficiency virus (HIV) which causes acquired immune deficiency syndrome (AIDS), hepatitis virus or the etiological agent of other diseases, the contaminated sharp can pose a serious threat to others who come in contact with it. There are many documented instances of HIV infection of medical workers who have been accidentally stabbed by a contaminated sharp.

Disposal of sharps is also a major problem. Disposal of contaminated materials poses both a logistic and a financial burden on the end user, such as the medial institution. In the 1980s, numerous instances of improperly disposed biological wastes being washed up on public beaches occurred. The potential for others, such as intravenous drug users, to obtain improperly disposed needles is also problematic.

There exist additional drawbacks to the traditional method of puncturing the skin of a patient with a sharp instrument for the purpose of drawing fluids or gases. Often, the stabbing procedure must be repeated, often resulting in significant stress and anxiety in the patient. The pain associated with being stabbed by a sharp instrument can be traumatizing, especially in pediatric patients. This can also cause significant stress and anxiety in the patient.

Clearly the current procedure for puncturing skin for the purpose of drawing fluids or gases has significant inherent problems. These problems arise because a sharp instrument is used in the procedure. Thus, there has existed a need for techniques to remove biomolecules, fluids or gases, and to administer pharmaceutical agents, which do not use a sharp instrument. Such methods would obviate the need for disposal of contaminated instruments, and reduce the risk of cross infection.

Lasers have been used in recent years as a very efficient and precise tool in a variety of surgical procedures. Among potential new sources of laser radiation, the rare-earth elements are of major interest for medicine. The most promising of these is a YAG (yttrium, aluminum, garnet) crystal doped with erbium (Er) ions. With the use of this crystal, it is possible to build an Erbium:YAG (Er:YAG) laser which can be configured to emit electromagnetic energy at a wavelength (2.94 microns) which is strongly absorbed by water. When tissue, which consists mostly of water, is irradiated with radiation at or near this wavelength, it is rapidly heated. If the intensity of the radiation is sufficient, the heating is rapid enough to cause the vaporization of tissue. Some medical uses of Er:YAG have been described in the health-care disciplines of dentistry, gynaecology and ophthalmology. Reference is made, for example, to Bogdasarov, B. V., et al., “The Effect of YAG:Er Laser Radiation on Solid and Soft Tissues”, Preprint 266, Institute of General Physics, Moscow, 1987; and Bol'shakov, E. N. et al., “Experimental Grounds for YAG:Er Laser Application to Dentistry”, SPIE 1353:160-169, Lasers and Medicine (1989).

Er:YAG lasers, along with other solid state lasers often employ a polished barrel crystal element such as a polished rod. A laser built with such a polished element maximizes the laser's energy output. Other lasers employ an entirely frosted element, normally with matte of about 50-55 microinch. However, in both cases, the energy output is typically separated into a central output beam surrounded by halo rays, or has an otherwise undesirable mode. Since it is extremely difficult to focus halo rays to a specific spot, the laser output may be unacceptable for specific applications.

Solid state lasers also typically employ two optic elements in connection with the crystal element. The optic elements consist of the rear (high reflectance) mirror and the front partial reflectance mirror, also known as an output coupler. The crystal element and the optic elements are rigidly mounted in order to preserve the alignment between them. However, changes in temperature, such as that caused by expansion of the crystal rod during flash lamp exposure, also cause shifts in alignment between the mirrors and the crystals. The misalignment of the mirrors and the crystal element results in laser output energy loss. Thus, the rigidly mounted elements require constant adjustment and maintenance. Moreover, thermal expansion of the crystal element during lasing can cause the crystal to break while it is rigidly attached to a surface with different expansion characteristics.

The use of a laser to perforate, ablate or alter one or more layers of the skin of a patient in order to remove biomolecules, fluids or gases, or to administer pharmaceutical substances has been proposed, in for example, U.S. Ser. No. 08/885,477 which is incorporated herein by reference. In that application, the alteration of a patient's skin is achieved by irradiating the surface of the skin by a pulse of electromagnetic energy emitted by a laser. Permeability of the stratum corneum may therefore be enhanced without ablation (vaporization) or perforation of tissue, or alternatively by ablating or perforating the stratum corneum. U.S. Ser. No. 08/885,477 also suggests that it is possible to very precisely alter skin or permeability thereof to a selectable extent without causing clinically relevant damage to healthy proximal tissue. The depth and extent of alteration may be accomplished by a judicious selection of the following irradiation parameters: wavelength, energy fluence (determined by dividing the energy of the pulse by the area irradiated), pulse temporal width and irradiation spot size.

Advantageously, the present invention avoids the use of sharps such as needles, conventionally used for sample extraction, thus substantially eliminating the risk of accidental injury to the health care worker, the patient, and anyone who may come into contact with the sharp, whether by accident or by necessity.

The invention advantageously also provides a safe and effective means for sampling of fluids, gases and/or biomolecules from the body and diagnostic testing of the sample at least in some embodiments in a single step. Furthermore, the invention advantageously avoids any contamination of the sample taken prior to testing of the sample, and avoids contact of the sample with the health care worker conducting the sampling procedure.

Still further, the invention advantageously provides a device which is portable and which may be operated under battery power, and which may be operated by the person on which the diagnosis is being conducted.

Advantageously the invention also minimizes any discomfort experienced by the person on which the diagnosis is being performed.

For the purpose of this application, “perforation” will mean only the complete ablation of all layers of the stratum corneum to reduce or eliminate its barrier function. “Ablation” may mean, depending upon the context, either partial ablation whereby less than all layers of the stratum corneum are ablated or perforated ablation.

Certain alterations of molecules in the stratum corneum or interstitial spaces may also occur without actual ablation, and this will result in enhanced permeation of substances into or out of the body through the skin. For the purpose of this application, the terms “irradiation” or “alteration”, or a derivative thereof, will generally mean perforation, ablation or modification which results in enhanced transdermal permeation of substances.

The mechanism for non-ablative alteration of the stratum corneum is not certain. It may involve changes in lipid or protein nature or function or from desiccation of the skin. Regardless, laser-induced alteration changes the permeability parameters of the skin in a manner which allows for increased passage of fluids and gases across the stratum corneum. For example, a pulse or pulses of infrared laser irradiation at a subablative energy of, for example, 60 mJ per 2 mm spot, reduces or eliminates the barrier function of the stratum corneum and increases permeability without actually ablating or perforating the stratum corneum itself. The technique may be used for transdermal delivery of drugs or other substances, or for obtaining samples of biomolecules, fluids or gases from the body. Different wavelengths of laser radiation and energy levels less than or greater than 60 mJ may also produce the enhanced permeability effects without ablating the skin.

Generally, the present invention relates to a diagnostic device for collecting and analysing a biological sample comprising:

an energy source providing means for perforating, ablating and/or altering the stratum corneum of an area of skin from which the biological sample is to be collected;

collection means for collecting the biological sample during or subsequent to perforation, ablation and/or alteration of the stratum corneum; and

analysing means for conducting diagnostic analysis of the collected sample.

According to one particular aspect of the invention there is provided a diagnostic device for collecting and analysing a biological sample comprising:

an energy source providing means for perforating, ablating and/or altering the stratum corneum of an area of skin from which the biological sample is to be collected;

a housing for receiving at least one test strip, the test strip being adapted to collect biological sample from the perforated, ablated and/or altered area of skin; and

analysing means for conducting diagnostic analysis of the collected sample.

According to this aspect, the device is provided with a housing for receiving at least one test strip. In a preferred embodiment, however, the housing is adapted to receive a cassette which includes a plurality of test strips, each of which is adapted to collect biological sample. The test strips are preferably consatinered within the cartridge, each test strip being fed through an aperture in the cartridge for use as desired. The device itself may also be provided with a guide or guides for guiding the test strips into a desired position for collection of biological sample. The device may further be provided with means for deactivating the device until a test strip is suitably positioned on the device. The feeding of the test strips may be manual or automated. Generally, feeding of the tape will be facilitated by a feeding mechanism within the device.

In a particularly preferred embodiment, the test strips are mounted on a continuous tape, preferably a tape formed from a barrier-type material such as Teflon. More preferably the test strips are spaced apart on the tape such that when housed within the cartridge, sections of the tape which do not have a test strip applied thereto are interposed between adjacent test strips and thereby act as a protective barrier, preventing biological cross-contamination between the test strips. Preferably, perforations are provided between individual test strips so that after use a test strip may be removed from the continuous tape and disgarded.

The test strips are preferably designed to facilitate transmission of the energy source through the test strip. For example, where laser ablation technology is employed and the energy source includes a laser, the test strips preferably include a transmission window to facilitate transmission of the laser through the test strip to the area of skin to be perforated, ablated and/or altered. The laser, or other energy source, preferably passes through the transmission window with minimal aberrations and losses. This may be achieved, for example, where the transmission window includes a Teflon film window. Also, the transmission window, for example of Teflon film, preferably acts as a protective barrier alleviating or preventing any biological splash-back of ablated material contaminating the device.

To facilitate collection of the biological sample, for example interstitial fluid, each test strip preferably includes a portion of absorbent material. Most preferably, the absorbent material portion is coincident with the transmission window discussed above. In this case, in a particular embodiment taken for exemplification, interstitial fluid which permeates the skin following perforation, ablation or alteration by application of a laser through a transmission window of the test strip is absorbed by the absorbent portion of the test strip without any repositioning of the device. The method for collection of the sample on the test strip may, however, vary depending on the nature of the biological sample to be collected. For example, the form of the test strip may be adapted for the collection of biomolecules from the surface of the skin to which the energy source, for example laser, has been applied or gases which permeate the treated skin. However, according to the particularly preferred form of the invention according to this aspect wherein interstitial fluid is collected using an absorbent portion of the test strip, each of the test strips is preferably provided with a chamber which is in fluid communication with the absorbent portion, for example by means of a capillary, and which receives the interstitial fluid. Most preferably, the chamber takes the form of a testing portion which constitutes the analysing means of the device. In particular, the chamber may include an optical or electrical system, or a combination thereof for conducting a diagnostic analysis on the collected sample. For example an optical system may include an optical colour change system and an electrical system may include an electrical contact incorporated into the test strip.

The above mentioned test strip or test strip cartridge may constitute the collection means, and optionally the testing means of the device as generally described above. Such a cartridge provides substantial advantages to the device according to this aspect of the invention.

In a preferred embodiment the cartridge is encoded and therefor may act as a calibrating device for calibration of the diagnostic device before or during use thereof. In a particularly preferred embodiment, the cartridge includes a micro-PCB which contains a calibration code and an identification number for the cartridge. In this case, the diagnostic device includes means for reading the encoded cartridge.

Accordingly, in another aspect of the invention there is provided a cartridge which includes a plurality of test strips, the cartridge and test strips being as described in the preceding paragraphs. That is, the invention also relates to a cartridge containing a plurality of test strips for collecting a biological sample, each test strip comprising an absorbent portion for absorbing the biological sample at an area of skin which has had applied thereto an energy source to perforate, ablate or alter the stratum corneum of the skin, wherein said test strips are adapted to facilitate transmission of the energy source to the skin, preferably by means of a transmission window coincident with or in the vicinity of the absorbent portion which allows transmission of the energy source to the skin. Preferred embodiments of this aspect of the invention will be appreciated from the above description.

The above described test strip application advantageously provides the diagnostic device with “single-step” diagnostic testing. That is, an operator of the device simply places the device in position and engages the device. The perforation, ablation and/or alteration of the stratum corneum and subsequent collection of sample and testing of the sample is automated and advantageously requires no further action by the operator of the device.

In an alternative embodiment, a “two-step” procedure is envisaged. In that case, the housing is again adapted to receive a plurality of test strips. In this case, however, the strips are provided in the form of a disc, each test strip being housed within a receptacle on the disc. The disc is, in use, rotatably mounted within the diagnostic device, each test strip being rotated into place for collection of sample as desired.

The two-step operation according to this embodiment involves a first step of applying energy to an area of skin to perforate, ablate or alter the stratum corneum. During the first step, the test strip is in a position remote from the area being treated. Following this, in a second step, a test strip is dislodged or ejected from its receptacle into a position to collect biological sample from the treated area of skin. The collection may be as discussed above, and similarly preferably involves the collection of interstitial fluid. In this case, however, the test strip preferably employs capillary action for collection of the fluid. More particularly, each test strip has a multi-layer structure including a base layer, preferably formed from plastic, P.C. electronic tracks which lead to electrical contacts within the diagnostic device, and an upper domed layer which creates the capillary action within the test strip. Generally, all of these layers will be laminated together.

The device preferably includes means for monitoring the amount of fluid being collected in the test strip. In this embodiment, as the fluid is drawn into the test strip, the amount of fluid is monitored and take up is continued until sufficient fluid is collected. Analysis is only conducted when sufficient interstitial fluid has been collected.

With regards to analysis of the interstitial fluid, taking glucose analysis as a specific example, two methods are generally used in blood glucose meters: color reflectance and sensor technology.

In color reflectance, or reflectance photometry, a drop of blood is placed on the strip. Glucose in the blood is oxidized enzymatically and then coupled with reduced chromogen to produce a color change in the strip. The color change is proportional to the amount of glucose present in the drop of blood. The meter quantifies the color change and generates a numerical value representative of the concentration of glucose present in the drop of blood. The darker the color, the higher the concentration of glucose in the sample.

Sensor technology meters use an electrochemical process to determine the glucose concentration. Again, a drop of blood is placed on the test strip, and the glucose contained within the drop is oxidized enzymatically. An electrode quantifies the electrical charge generated by this reaction and displays a numerical value representative of the concentration of glucose present in the drop of blood. Sensor meters are generally considered second-generation meters. It is here where technology is again influencing the way patients participate in SMBG.

Sensor meters may also be classified based on the electrochemical principle employed, that is amperometry or coulometry.

Amperometric meters use an electrochemical reaction, which in the presence of an applied potential results in electron transfer and generation of an electrical current that is proportional to the concentration of glucose. This system measures a small percent of glucose and produces an electrochemical response curve that may be affected by the same factors that affect reflector meters: environmental temperature and variations in hematocrit. These factors may change the shape of the response curve and interfere with the accuracy of the glucose measurement. Also, this method is difficult to adapt to small blood samples because only a portion of the glucose is used to generate the electrochemical signal, and with small samples the signal will be weak. Therefore, amperometry requires a sufficient drop of blood to produce an accurate reading.

Coulometric meters, the newest technology on the market, use an electrochemical reaction whereby the total accumulated charge of the reaction is in proportion to the glucose concentration. In this system, all glucose is consumed and measured. In other words, coulometric meters convert the entire glucose content of a blood sample into an electric charge. Coulometric meters produce a response curve, but the total charge or area under the curve is used to calculate the glucose concentration. Factors such as environmental temperature and hematocrit may alter the shape of the response curve, but do not alter the area under the curve. Therefore, glucose measurements are unaffected by these factors. The principle of coulometry limits the effect of environmental temperature and variations in hematocrit. This method is ideal for small analyte samples because by converting all glucose present into a charge, the signal is stronger, and far less blood is required to produce an accurate reading of the corresponding glucose concentration.

Blood glucose and interstitial fluid glucose levels are essentially equal when blood glucose is not changing rapidly (e.g. fasting glucose levels). However, rapidly changing glucose levels (after a high caloric meal, or after a high insulin dose) create a lag between blood and interstitial fluid measurements. The differences between the measurements in these fluids at this lag time do not affect the clinical utility of an interstitial fluid monitoring device because they are minor (lag usually only lasts 10 minutes) and because the data is analyzed in such a way that minor differences are negligible. Also, it has been shown that glucose levels in interstitial fluid actually drop before blood glucose and this would mean interstitial fluid monitoring would allow an impending hypoglycemic episode to be detected earlier than with blood monitoring. This is believed to be advantageous with regard to this particular area of application of the device of the invention.

Analysis is preferably achieved electronically using an electrical system within the diagnostic device. That is, the electronic tracks of the test strip advantageously engage electrical contacts within the device to facilitate analysis of the fluid. The electrical contacts may also assist in holding the test strip in position during collection of the interstitial fluid.

The device preferably includes a mechanism for ejecting used test strips after testing is complete. The mechanism may be manual or automatic and preferably ejects the used test strip through a port in the device.

The disc may be encoded to facilitate calibration of the device. As such, the device may include means for reading the encoded disc, or may include input means for inputting relevant identification data which may be printed on the disc.

A laser can be used to perforate or alter the skin through the outer surface, such as the stratum corneum layer, but not as deep as the capillary layer, to allow the collection of biomolecules, fluids or gases as discussed above. Although the most preferred forms of collection have been described, it should be recognised that more active collection methods may utilize electrical gradients, vacuum or suction pressure, or a variety of other active transport methods. For example, in order to facilitate an electrical gradient for the purpose of capturing biomolecules from within a subject, the same procedure as is used in iontophoretic delivery of a particular substance may be used, except that the polarity of the electrodes used to establish the gradient are reversed. The present invention includes methods of collecting at least one substance from within a subject, comprising administering an amount of energy to a portion of skin sufficient to cause alteration at the energized site, at least as deep as the outermost surface of the stratum corneum, and collecting said substance from said energized site.

Once the desired substances have permeated through the skin, there are several means of capturing the substances for collection and analysis. Such capture means includes medium selected from the group consisting of gel, viscous materials, activated carbon or other adsorbant material such as ceramic, and activated carbon; alternatively, absorbent medium such as patch or dressing materials may offer capture means. It should be understood that means for facilitating such collection may also be provided in the diagnostic device of the invention.

The collected substances may be used for a wide variety of tests. For example, the technique of the present invention may be used to sample extracellular fluid in order to quantify glucose or the like. Glucose is present in the extracellular fluid in the same concentration as (or in a known proportion to) the glucose level in blood (Lonnroth P., Strinberg L., “Validation of the “internal reference technique” for calibrating microdialysis catheters in situ.” Acta Physiological Scandinavica 153(4):37580, 1995 Apr.)

Also, HIV is present extracellularly and it is obvious that there is a benefit to obtaining samples for HIV analysis without having to draw blood with a sharp that could subsequently contaminate the health-care provider.

The energy source may include any suitable means provided that perforation, ablation and/or alteration of the stratum corneum can be achieved. Various preferred options will be dealt with herebelow. However, it should be recognised that other forms of energy, including mechanical, may be used in particular instances without departing from the invention.

The practice of the present invention has been found to be effectively performed by various types of lasers; for example, the Venisect, Inc., Er:YAG laser skin perforator, or the Schwartz: Electro-Optical Ho:YAG. Any pulsed or gated continuous wave laser producing energy that is strongly absorbed in tissue may be used in the practice of the present invention to produce the same result at a non-ablative wavelength, pulse length, pulse energy, pulse number, and pulse rate.

The Er:YAG lasing material is a preferred material for the laser used in accordance with the present invention because the wavelength of the electromagnetic energy emitted by this laser, 2.94 microns, is very near one of the peak absorption wavelengths (approximately 3 microns) of water. Thus, this wavelength is strongly absorbed by water and tissue. The rapid heating of water and tissue causes ablation or alteration of the skin.

Other useful lasing material is any material which, when induced to lase, emits a wavelength that is strongly absorbed by tissue, such as through absorption by water, nucleic acids, proteins or lipids, and consequently causes the required perforation, ablation or alteration of the skin. A laser can effectively cut or alter tissue to create the desired ablations or alterations where tissue exhibits an absorption coefficient in the range of between about 10 to 10,000 cm⁻¹. Examples of useful lasing elements are pulsed CO₂ lasers, Ho:YAG (holmium:YAG), Er: YAP, Er/Cr:YSGG (erbium/chromium: yttrium, scandium, gallium, garnet; 2.796 microns), Ho:YSGG (holmium:YSGG; 2.088 microns), Er:GGSG (erbium: gadolinium, gallium, scandium, garnet), Er:YLF (erbium: yttrium, lithium, fluoride; 2.8 microns), Tm:YAG (thulium: YAG; 2.01 microns), Ho:YAG (holmium: YAG; 2.127 microns); Ho/Nd:YAlO₃ (holmium/neodymium: yttrium, aluminate; 2.85-2.92 microns), cobalt:MgF2 (cobalt: magnesium fluoride; 1.75-2.5 microns), HF chemical (hydrogen fluoride; 2.6-3 microns), DF chemical (deuterium fluoride; 3.6-4 microns), carbon monoxide (5-6 microns), deep UV lasers, diode lasers and frequency tripled Nd:YAG (neodymium:YAG, where the laser beam is passed through crystals which cause the frequency to be tripled). The traits common to all such lasing elements, justifying inclusion of each such element in this group, are that they are all capable of transmitting energy to the skin in the amounts and manner necessary to either reduce the electrical impedance of the skin or otherwise enhance permeation.

Utilizing current technology, some of these laser materials provide the added benefit of small size, allowing the laser to be small and portable. In addition to Er:YAG, Ho:YAG or Er:YSGG lasers provide this advantage.

Optionally, the beam can be broadened, for instance though the use of a concave diverging lens, prior to focusing through the focusing lens. This broadening of the beam results in a laser beam with an even lower energy fluence rate a short distance beyond the focal point, consequently reducing the hazard level. Furthermore, this optical arrangement reduces the optical aberrations in the laser spot at the treatment position, consequently resulting in a more precise ablation or alteration. Also optionally, the beam can be split by means of a beam-splitter to create multiple beams capable of ablating or altering several sites simultaneously or nearly simultaneously.

In addition to the pulsed lasers listed above, a modulated laser can be used to duplicate a pulsed laser.

If the laser energy is not strongly absorbed in the tissue, a dye that absorbs said energy can be applied on, in or under the skin prior to application of the laser thereto. As such, the diagnostic device may include means for applying a dye to the area of skin to be treated or being treated.

In another embodiment of the invention, the energy source includes radiofrequency or microwave energy which is applied directly to the surface of the tissue, or to a target adjacent to the tissue, in such a way that the epithelial layers of the tissue are altered to make the layers “leaky”. In the case of skin, the stratum corneum may be ablated through the application of electromagnetic energy to generate heat. Alternatively, shear forces may be created by targeting this energy on an absorber adjacent to the skin, which transfers energy to create stress waves that alter or ablate the stratum corneum. It is a specific embodiment of this invention that radiofrequencies producing a desired rapid heating effect, localized on stratum corneum, result in an ablative event, while minimizing coagulation. This removal of the stratum corneum in this way will result in increased permeability across the compromised tissue interface.

Alternatively, delivery of electromagnetic energy at these wavelength may be optimized, by adjusting pulse duration, dwell time between pulses, and power to result in a rapid, intermittent excitation of molecules in the tissues of interest, such that there is no net coagulation effect from heating, but molecules are altered transiently to effect a transient change in membrane conformation that results in greater “leakiness”. It is a further embodiment of the invention to continuously apply energy with the appropriate energy and pulse mode characteristics so that these transient alterations are maintained as long as the energy cycle is applied, thus creating a means for maintaining increased membrane permeability over time.

In another embodiment, a “leaky” membrane or ablation site in skin may be created by first applying electromagnetic energy, including light, microwave or radiofrequency, such that membrane or intramembrane structures are realigned, or the membrane is compromised otherwise, so as to improve permeation. This step is followed by application of electromagnetic energy induced pressure to drive molecules across tissue interfaces and between cellular junctions at a greater rate than can be achieved by either method alone. The laser energy may be delivered continuously or in discrete pulses to prevent closure of the pore. Optionally, a different wavelength laser than is used to create the pore may be used in tandem to pump molecules through the pore. Alternatively a single laser may be modulated such that pulse width and energy vary and alternate over time to alternatively create a pore through which the subsequent pulse drives the molecule. As such, the diagnostic device may include a combination of energy sources to facilitate this embodiment.

In one embodiment, laser energy is directed through optical fibers or guided through a series of optics provided by the diagnostic device such that pressure waves are generated which come in contact with or create a gradient across the membrane surface. These pressure waves may be optionally used to create a pressure gradient such that the pressure waves facilitate permeation of, for example, interstitial fluid through the treated area.

In order to sterilize the skin before perforation, ablation or alteration, a sterile alcohol-impregnated patch of paper or other thin material can optionally be placed over the site to be ablated. This material can also prevent the blowing off of potentially infected tissue in the plume released by the ablation. The material must be transparent to the energy source, for example the laser beam. Examples of such material are a thin layer of quartz, mica, or sapphire. Alternatively, a thin layer of plastic, such as a film of polyvinyl chloride, can be placed over the skin. Although the laser beam will perforate the plastic, the plastic prevents most of the plume from flying out and thus decreases any potential risk of contamination from infected tissue. Additionally, a layer of a viscous sterile substance such as vaseline can be added to the transparent material or plastic film to increase adherence of the material or plastic to the skin and further decrease plume contamination. In this regard, the diagnostic device may be provided with an applicator for applying material or solution to the area of skin to be treated for sterilization purposes, or for any other purpose as desired.

Reference will now be made to the accompanying drawings which illustrate preferred embodiments of the present invention and in which:

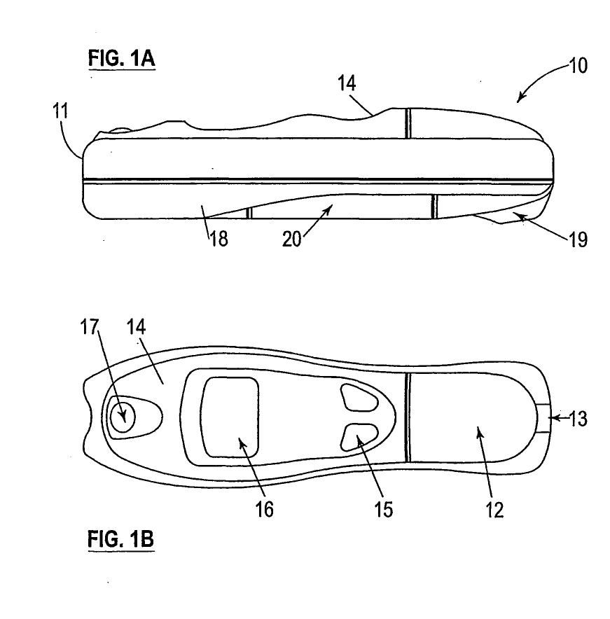

FIGS. 1A-1B illustrate a diagnostic device according to one aspect of the invention;

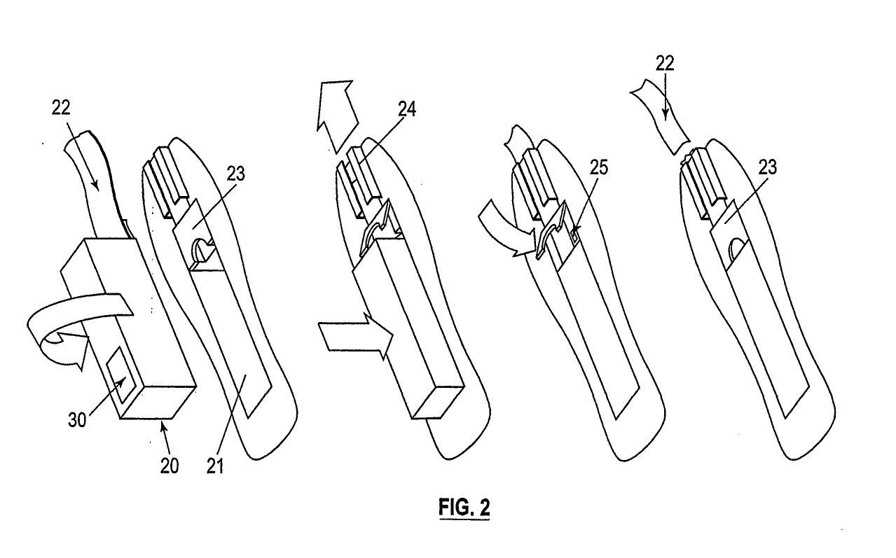

FIG. 2 illustrates insertion of a test strip cartridge into the diagnostic device of FIGS. 1A, 1B;

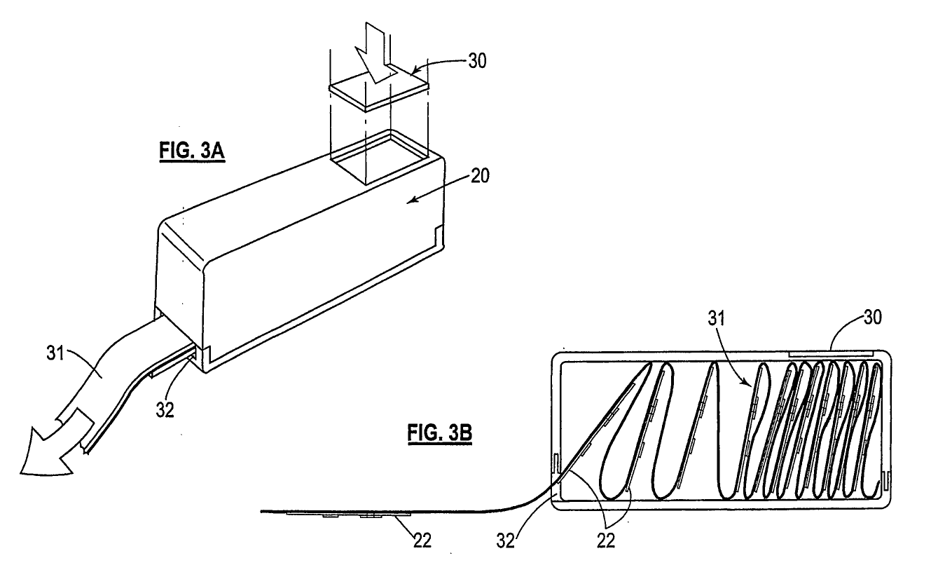

FIGS. 3A-3B illustrate views of the test strip cartridge;

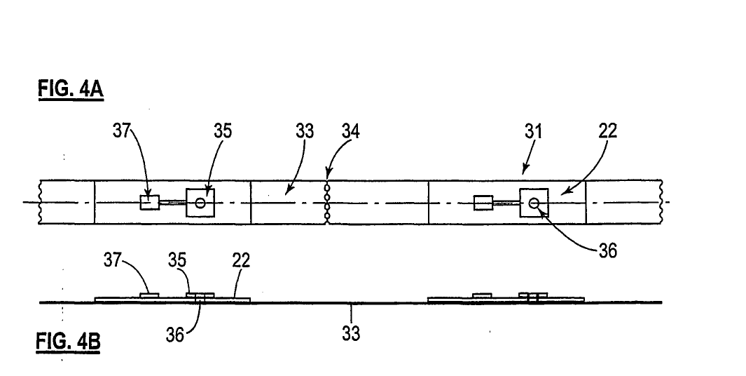

FIGS. 4A-4B illustrate a tape which includes the test strips;

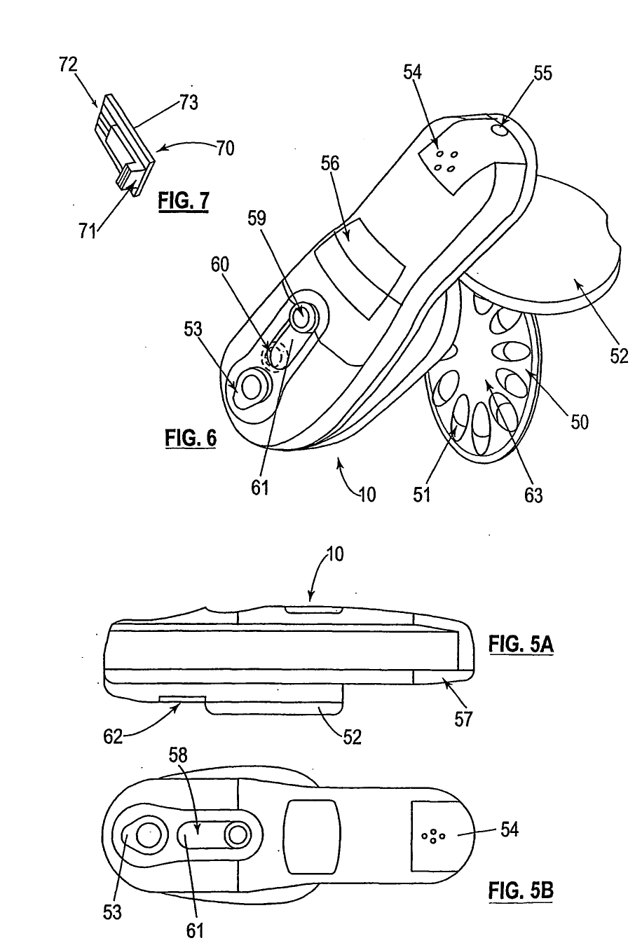

FIGS. 5A-5B illustrate a second embodiment of the diagnostic device;

FIG. 6 illustrates the diagnostic device of FIGS. 5A-5B and a disc including a number of test strips; and

FIG. 7 illustrates a test strip removed from the disc illustrated in FIG. 6.

For convenience, the diagnostic device illustrated will hereinafter be referred to as a glucometer adapted for laser perforation, ablation or alteration of the stratum corneum. However, it should be recognised that various modifications to the illustrated devices may be possible.

Referring to FIGS. 1A-1B, a glucometer 10 includes a housing 11 for housing the componentry of the glucometer 10. The housing is formed from a resilient material, such as a resilient plastic material, for example by injection molding or the like. Componentry housed by the housing 11 may include a laser diode, laser electronics, a power source such as a battery and various other componentry as desired. The battery power source 12 is represented in FIG. 1B. The housing 11 is provided with a charger jack 13 for recharging the battery 12.

The external configuration of the glucometer 10 is molded to provide a contoured appearance. The upper side 14 of the glucometer 10 is provided with on/off buttons 15 and an LCD display 16. The LCD display 16 may provide an operator of the glucometer 10 with relevant information relating to the collection of biological samples such as interstitial fluid and the results of diagnostic analysis made on the sample. A laser fire button 17 is also provided which can be engaged by the operator when the glucometer 10 is positioned over an area of skin to be perforated, ablated or altered.

The underside 18 of the glucometer 10 includes a dye patch applicator 19 for applying a dye to the skin in order to amplify the laser efficacy for improved ablation. The dye may include any material which aids in the laser perforation, ablation or alteration of the stratum corneum.

A test strip cartridge 20 is housed in a housing 21 on the underside 18 of the glucometer 10. A test strip 22 is fed from the cartridge 20 in use by an automatic mechanism. The cartridge 20 is clipped in place in the housing 21 by means of a clip 23. The underside 18 of the glucometer 10 is provided with a guide 24 through which the test strip 22 is fed to ensure that the test strip is positioned over the area of skin to be tested in an appropriate fashion. In this regard, the glucometer 10 may also be provided with a number of safety mechanisms 25 incorporated into the design of the glucometer 10 to prevent operation of the unit or firing of the laser unless the cartridge 20 is correctly loaded into the glucometer 10, or unless the test strip 22 is correctly positioned for diagnosis.

Referring to FIGS. 3A-3B and FIGS. 4A-4B, the cartridge 20 is provided with a micro-PCB which contains a calibration code and identification number for the cartridge 20. A tape 31 on which is mounted a plurality of test strips 22 is fed through an aperture 32 in the cartridge 20 for diagnosis. The test strips 22 may be applied to the tape 31 by any suitable means, such as via an adhesive. As can be seen in FIG. 3B, the tape 31 to which have been applied a plurality of test strips 22 in a concertina within the cartridge 20. This advantageously facilitates multiple analysis to be carried out without replacement of the cartridge 20.

The tape 31 itself is formed from an appropriate material such as Teflon so that when positioned in the cartridge, a film of Teflon is interposed between adjacent test strips 22. This ensures that contamination of subsequent test strips 22 is avoided in use. The lengths of Teflon 33 of the tape 31 are provided with perforations 34 so that each test strip 22 may be removed from the tape 31 and discarded after use.

Each test strip 22 includes an absorbent portion 35 of porous material which absorbs interstitial fluid permeating through the skin following perforation, ablation or alteration. The absorbent portion 35 further includes a transmission window 36 which is adapted to transmit laser energy. The transmission window includes a Teflon film through which the laser beam passes with minimal aberrations and losses. The inclusion of a Teflon film in the window 36 provides a protective barrier and advantageously prevents any biological splash back entering and contaminating the glucometer 10 when the skin is perforated, ablated or altered. Furthermore, the disposable Teflon-backed tape 31 advantageously protects multiple users from cross-contamination from ablated skin waste.

Each test strip 22 further includes a compartment 37 which is in fluid communication with the absorbent portion 35 and which, therefore receives interstitial fluid from the absorbent portion 35. Analysis of the interstitial fluid is conducted in the compartment 37 by means of electronic or colour change systems which are provided by the glucometer 10.

Referring to FIGS. 5A-5B and FIG. 6, in an alterative embodiment the glucometer 10 is adapted to house a disc 50 which includes a plurality of receptacles 51, each of which contains a test strip 70 (illustrated in FIG. 7). In this embodiment, a disc housing is provided with a closure 52 which secures the disc in place in the disc housing. Again, the glucometer 10 is provided with an on/off switch 53 which in this case also acts as the laser fire switch. There is also provided a battery power source 54 and a charger jack 55 for recharging the battery. An LCD display 56 and dye patch applicator 57 are also provided.

In this case, a two-step analytical diagnosis is envisaged whereby a laser is initially applied to the skin to perforate, ablate or alter the stratum corneum and to facilitate permeation of interstitial fluid through the skin. Subsequent to this, an individual test strip 70 is ejected from the receptacle 51 of the disc 50 in which it is housed so that the test strip 70 comes in contact with the interstitial fluid. Ejection of the test strip is facilitated by an ejection mechanism 58 which is operable by sliding a button 59 on the upper side of the glucometer 10 from a first position toward the on/off switch 53 to a second position 60 (illustrated in FIG. 6) along a slot 61.

The glucometer 10 is further provided with a laser aperture 62 through which a laser beam 15 passes. The laser aperture 62 advantageously includes a protective barrier such as a Teflon lens which prevents any biological splash back from gathering on, in or around the laser aperture 62.

In this embodiment, the disc 50 is advantageously provided with a printed identification number 63 which may be used for calibration of the glucometer 10. That is, this number may be programmed into the glucometer 10 once the glucometer is turned on to effect calibration of the unit. Each of the test strips 70 of the disc 50 includes a capillary 71 for siphoning of interstitial fluid from the surface of the perforated, ablated or altered skin. The capillary is in fluid communication with electronic sensors 72 on the end of the test strip 70. The electrical sensors 72 sense when a sufficient quantity of interstitial fluid has been collected and, at that time, analysis of the fluid is initiated. In this regard, the electronic sensors are advantageously in electrical contact with contacts housed within the glucometer 10 providing a means to analyse the interstitial fluid, and also acting to hold the test strip 70 in place during the testing procedure. The test strip 70, therefore, is formed as a multi-layer structure including a plastic base 73, the electrical sensors 72 and the capillary 71.

In use, the diagnostic device is applied to the skin surface at a desired area to be perforated, ablated or altered. A dye patch is applied to the area of skin, if desired, prior to firing of the energy source, for example the laser, onto the skin.

The area of skin on which the analysis is performed is advantageously uniform in thickness and easily accessible, such as the forearm or the thigh. The area of ablation or alteration is generally approximately 400 microns in diameter and between 20-100 microns in depth. At this depth, interstitial fluid is easily accessible and permeable through the skin.

Advantageously, the device according to the invention makes it possible to conduct an analysis on a biological sample in a single or minimal steps without requiring repositioning of the device during the analysis.

Still further, it is envisaged that the device may further include means for administering a pharmaceutically active substance to a patient following, and in response to, the results of the diagnostic analysis conducted. More particularly, it is envisaged that the delivery of the pharmaceutical substance through the skin will be substantially enhanced due to the perforation, ablation or alteration which has been made to the stratum corneam. As such, any suitable means may be provided to facilitate administration of the pharmaceutical substance through the treated area of skin, preferably without repositioning of the device.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference to any prior art in this specification is not, and should not be taken as, an acknowledgment or any form of suggestion that that prior art forms part of the common general knowledge in Australia.

Those skilled in the art will appreciate that the invention described herein is susceptible to variations and modifications other than those specifically described. It is to be understood that the invention includes all such variations and modifications which fall within its spirit and scope. The invention also includes all the steps, features, compositions and compounds referred to or indicated in this specification, individually or collectively, and any and all combinations of any two or more of said steps or features.

1-18. (Canceled)

19. A device, comprising:
a housing;
a laser source mounted in the housing for emitting a laser beam onto the skin of a patient with sufficient energy to enhance the permeability of the skin by perforating, ablating or altering the skin;
capturing media for capturing bodily fluids released through the skin; and
a container mounted on the housing for storing and dispensing the capturing media from therein.

20. The device of claim 19 wherein the container is adapted for detachable coupling to the housing.

21. The device of claim 19 wherein the container is rotatably attached to the housing.

22. The device of claim 21 wherein the container comprises one or more openings for dispensing the capturing media.

23. The device of claim 19 wherein the capturing media is absorbent.

24. The device of claim 19 wherein the capturing media is adsorbent.

25. The device of claim 19, wherein the capturing media comprises:
a continuous strip; and
a plurality of capturing portions mounted on the strip for capturing bodily fluids released through the skin.

26. The device of claim 25, wherein each capturing portion comprises:
a transparent portion for transmitting a substantial portion of the laser beam therethrough.

27. The device of claim 19, wherein the container comprises:
a plurality of receptacles, each receptacle for storing and dispensing a piece of capturing media from therein.

28. The device of claim 19, comprising:
an analyzer mounted in the housing for analyzing bodily fluids captured by the capturing media.

29. The device of claim 28, wherein the analyzer comprises
a glucometer.

30. The device of claim 28, comprising
an analyzer mounted in the housing for analyzing bodily fluids captured by the capturing media; and wherein
the plurality of capturing media each comprise electronic sensors for interfacing with the analyzer to analyze the retained bodily fluids.

31. The device of claim 19 further comprising
a detachable casette reversibly mounted on the housing; and
test strips within said casette to accept bodily fluids.

32. The device of claim 19 further comprising
a detachable, rotatable disc comprising a plurality of receptacles, reversibly mounted on the housing; and
a plurality of test strips, each located within a receptacle on the disc to accept bodily fluids.

33. The device of claim 19 further comprising
a detachable cartridge, reversibly mounted on the housing;
a continuous tape of test strips, within the cartridge, spaced on the tape so as to allow each test strip to accept a separate sample of bodily fluids.

34. The device of claim 33 wherein each test strip comprises
a laser transmitting window; and
an absorbent portion to accept bodily fluids.

35. The device of claim 33 wherein the test strip comprises
a chamber for the collection of bodily fluids; and
an absorbent portion which is in fluid communication with the chamber.
